(19) United States
(12) Patent Application Publication
(10) Pub. No.: US 2022/0387967 A1
(43) Pub. Date: Dec. 8, 2022

(54) MOLECULAR SIEVE ACTIVATED CARBON

(71) Applicant: OSAKA GAS CHEMICALS CO., LTD., Osaka-shi, Osaka (JP)

(72) Inventors: Natsuko KOJIMA, Osaka-shi, Osaka (JP); Yasuyuki YAMANE, Osaka-shi, Osaka (JP)

(21) Appl. No.: 17/775,811

(22) PCT Filed: Nov. 10, 2020
(86) PCT No.: PCT/JP2020/041963

§ 371 Date: May 10, 2022

(30) Foreign Application Priority Data
Nov. 13, 2019 (JP) 2019-205212

(51) Int. Cl. B01J 20/20; B01J 20/28; B01D 15/02; C12H 6/00

(52) U.S. Cl. CPC B01J 20/20; B01J 20/28019; B01J 20/2808; B01D 15/02; C12H 6/00; B01D 2253/102; B01D 2253/308; B01D 2253/34

(57) ABSTRACT

An carbon molecular sieve for adsorbing water molecules in an alcohol solution to separate an alcohol from the water molecules, wherein the total volume of pores with an inlet diameter of 0.33 nm or more as determined by a molecular probe method is not less than 3 times the total volume of pores with an inlet diameter of 0.46 nm or more. This carbon molecular sieve is an alcohol concentration material that is capable of efficiently concentrating alcohol without performing a distillation step and that is easily reusable.

### TECHNICAL FIELD

The present invention relates to a carbon molecular sieve.

### BACKGROUND ART

Biomass alcohol represented by bioethanol is usually produced by alcoholic fermentation from plant-derived raw materials and is attracting attention as carbon-neutral energy. In the case of bioethanol, for example, the concentration of ethanol collected after alcoholic fermentation is about 10 mass %; thus, the produced biomass alcohol must be subjected to a dehydration step by distillation etc. Biomass alcohol for use as a synthetic raw material, a fuel, and the like is required to have a concentration of 97 mass % or higher (preferably 99 mass % or higher). However, water and ethanol, in particular, form an azeotropic mixture when the ethanol concentration increases (e.g., 95 mass % or higher), and the ethanol concentration in the gas phase becomes equal to that in the liquid phase. The alcohol concentration thus cannot be further increased by distillation. This means that it is extremely difficult to increase the ethanol concentration in the liquid phase to 97 mass % or higher by typical methods such as distillation.

Known methods for further increasing the ethanol concentration include an azeotropic distillation method in which a third component such as benzene is added to forcibly change the composition of an azeotropic mixture. However, this method has a disadvantage in that the energy required for concentrating ethanol is extremely high. To reduce the energy required for concentrating ethanol, a method in which water is removed by a separation method that does not depend on vapor-liquid phase equilibrium is also known, such as a method in which water is removed from ethanol vapor after distillation using zeolite etc. (see, for example, Non-patent Literature (NPL) 1).

### CITATION LIST

### Non-Patent Literature

NPL 1: Food Technol. Biotechnol. 56(3) 289-311 (2018)

### SUMMARY OF INVENTION

### Technical Problem

However, the method in which water is removed from ethanol vapor after distillation using zeolite etc. does not sufficiently reduce the energy required for ethanol concentration because high-temperature heating is required to obtain ethanol vapor for contact with zeolites. Further, solid zeolite when used for dehydration is not reusable because desorption of water is difficult after ethanol concentration even with high-temperature heating (e.g., 200° C. or higher).

Although the above explanation is based on the case of bioethanol as an example, the same problems also apply to other alcohols.

Therefore, an object of the present invention is to provide an alcohol concentration material that is capable of efficiently concentrating alcohol without performing a distillation step and that is easily reusable.

### Solution to Problem

The present inventors conducted extensive research to achieve the above object, and consequently found that the use of a carbon molecular sieve with a specific pore-entrance size to adsorb water makes it possible to efficiently concentrate alcohol without performing a distillation step. The carbon molecular sieve is capable of desorption of the adsorbed water by mild heating (about 70 to 80° C.) and thus also has excellent reusability. Based on these findings, the present inventors conducted further research to complete the present invention. More specifically, the present invention encompasses the following embodiments.

Item 1. A carbon molecular sieve for adsorbing water molecules in an alcohol solution to separate an alcohol from the water molecules,

Wherein the total volume of pores with an inlet diameter of 0.33 nm or more as determined by a molecular probe method is not less than 3 times the total volume of pores with an inlet diameter of 0.46 nm or more.

Item 2. The carbon molecular sieve according to Item 1, which has a crushed shape, pellet shape, plate shape, rod shape, hollow shape, block shape, honeycomb shape, spherical shape, elliptical spherical shape, distorted shape, or fibrous shape.

Item 3. The carbon molecular sieve according to Item 2, wherein the pellet has a maximum diameter of 0.5 to 5.0 mm and an aspect ratio of 1:1 to 1:5.

Item 4. The carbon molecular sieve according to any one of Items 1 to 3, which is a carbide of at least one material selected from the group consisting of coal, coconut or palm shells, natural fibers, synthetic fibers, synthetic resins, and charcoal.

Item 5. The carbon molecular sieve according to any one of Items 1 to 4, wherein the alcohol is an alcohol having 1 to 6 carbon atoms.

Item 6. The carbon molecular sieve according to any one of Items 1 to 5, wherein the ½ equilibrium adsorption time of the water molecules is not more than ½ of the ½ equilibrium adsorption time of the alcohol.

Item 7. A water molecule adsorbent comprising the carbon molecular sieve of any one of Items 1 to 6.

Item 8. An alcohol concentration material comprising the carbon molecular sieve of any one of Items 1 to 6.

Item 9. An alcohol concentration device comprising the water molecule adsorbent of Item 7 or the alcohol concentration material of Item 8.

Item 10. A method of producing an alcohol with a concentration of 97 mass % or more, the method comprising bringing the carbon molecular sieve of any one of Items 1 to 6 into contact with an alcohol solution.

Item 11. A method for concentrating an alcohol, the method comprising bringing the carbon molecular sieve of any one of Items 1 to 6 into contact with an alcohol solution.

### Advantageous Effects of Invention

The carbon molecular sieve of the present invention is a material that is capable of efficiently concentrating alcohol without performing a distillation step and is easily reusable. Therefore, the carbon molecular sieve of the present invention can be suitably used for an alcohol concentration device.

### BRIEF DESCRIPTION OF DRAWINGS

FIG. 1 is a schematic diagram of an ethanol concentration device used in the ethanol concentration test of Test Examples 2 and 3.

FIG. 2 is a graph showing the results of the ethanol concentration test of Test Example 3.

FIG. 3 is a graph showing the results of the ethanol concentration test of Test Example 4.

FIG. 4 is a graph showing the results of the methanol concentration test of Test Example 5.

### DESCRIPTION OF EMBODIMENTS

In the present specification, the terms “contain,” “include,” and “comprise” encompass the concepts of comprising, consisting essentially of, and consisting of.

Further, in the present specification, numerical ranges represented by “A to B” mean A or more and B or less.

### 1. Carbon Molecular Sieve

The carbon molecular sieve of the present invention is used for adsorbing water molecules in an alcohol solution to separate an alcohol from the water molecules, wherein the total volume of pores with an inlet diameter of 0.33 nm or more as determined by a molecular probe method is not less than 3 times the total volume of pores with an inlet diameter of 0.46 nm or more.

In the present invention, the molecular probe method refers to a method for measuring adsorption amounts using several types of adsorbents (probe molecules) with different molecular diameters, and determining the pore diameter distribution based on the relationship between the molecular diameter and the pore volume. Probe molecules cannot enter pores that are smaller than their own size. Thus, the pore volume calculated from the adsorbed amount of each probe molecule corresponds to a volume of pores that are larger than the molecular diameter of the probe molecule used. The pore volumes corresponding to each probe molecule can be calculated from the adsorption amount and the liquid density of the molecule when the relative pressure (a value obtained by dividing the equilibrium pressure at the time of adsorption measurement by the saturated vapor pressure) is sufficiently high (e.g., about 0.9), and can be calculated from the adsorption isotherm according to the Dubinin-Astakhov equation even when the relative pressure is low.

In particular, in the present invention, probe molecules are adsorbed on the carbon molecular sieve in an atmosphere of 25° C. and 1 atm, and the total volume of pores with a specific inlet diameter can be calculated from the change in the mass of the carbon molecular sieve after equilibrium adsorption, as disclosed in T. A. Braymer et al., Carbon, Vol. 32, 445-452, 1994. For example, the total volume of pores with an inlet diameter of 0.33 nm or more and the total volume of pores with an inlet diameter of 0.46 nm or more can be calculated by using carbon dioxide (minimum molecular diameter: 0.33 nm) and chloroform (minimum molecular diameter: 0.46 nm), respectively, as molecular probes.

As described above, in the carbon molecular sieve of the present invention, the total volume of pores with an inlet diameter of 0.33 nm or more as determined by the molecular probe method is not less than 3 times, in particular, 5 to 100 times the total volume of pores with an inlet diameter of 0.46 nm or more. If the total volume of pores with an inlet diameter of 0.33 nm or more is less than 3 times the total volume of pores with an inlet diameter of 0.46 nm or more, an alcohol solution cannot be sufficiently concentrated.

The carbon molecular sieve of the present invention is capable of preferentially adsorbing water molecules from among alcohols and water molecules in an alcohol solution. More specifically, when the carbon molecular sieve of the present invention and an alcohol solution are brought into contact with each other, the adsorption rate of water molecules is significantly greater than the adsorption rate of alcohol. This enables water molecules to be preferentially adsorbed, and as a result, the alcohol solution is concentrated to increase the concentration to, for example, 97 mass % or higher, and particularly 99 mass % or higher.

In the carbon molecular sieve of the present invention, when the pore-entrance size as determined by the molecular probe method is within a predetermined range, the pore-entrance size of the carbon molecular sieve is sufficiently larger than the size of water molecules but is almost the same as or slightly smaller than the size of alcohol molecules. Therefore, the adsorption rate of water molecules is significantly greater than the adsorption rate of alcohol molecules. As a result, the alcohol is more easily concentrated by more efficiently desorbing water molecules from an alcohol solution without performing a distillation step, unlike in conventional methods. From the standpoint of appropriately adjusting the adsorption rate of water so that the amount of alcohol that can be treated per hour is within a more appropriate range, the pore-entrance size of the carbon molecular sieve is preferably greater than the size of water molecules. Therefore, in the carbon molecular sieve of the present invention, the pore-entrance size as determined by the molecular probe method is preferably 0.33 to 0.46 nm, and more preferably 0.35 to 0.40 nm, so as to significantly increase the adsorption rate of water molecules, compared with the adsorption rate of alcohol molecules, and consequently to more easily increase the concentration of alcohol by more efficiently desorbing water molecules from the alcohol solution without performing a distillation step, unlike in conventional methods.

In the carbon molecular sieve of the present invention, the pore-entrance size preferably has a size that allows water molecules to easily pass through but that does not allow alcohol to easily pass through, so that water molecules are adsorbed while alcohol is not easily adsorbed on the carbon molecular sieve of the present invention. From the standpoint of efficiently concentrating an alcohol solution, however, a greater pore volume is more preferred to adsorb a large number of water molecules. From this standpoint, the water vapor adsorption amount in terms of the pore volume of the carbon molecular sieve of the present invention is preferably 100 NmL/g or more, and more preferably 150 NmL/g.

The shape of the carbon molecular sieve of the present invention is not particularly limited. For example, the carbon molecular sieve of the present invention can have a shape that is applicable to known adsorbents. For example, the carbon molecular sieve of the present invention can have a crushed shape, a pellet shape, a plate shape, a rod shape, a hollow shape, a block shape, a honeycomb shape, a spherical shape, an elliptical spherical shape, a distorted shape, a fibrous shape, or the like. The carbon molecular sieve of the present invention preferably has a pellet shape from the standpoint of particularly easily concentrating alcohol, being easily processed, having high strength, having higher bulk density, and being easily applicable to various uses. When the carbon molecular sieve of the present invention is in the form of pellets, unnecessary fine powder is unlikely to be formed; thus, blockage of the piping etc. of a device is unlikely to occur in a dehydration step for alcohol.

The carbon molecular sieve of the present invention in the form of pellets may have a planar view shape that is, for example, applicable to known adsorbents. For example, the pellet may have a circular shape, an elliptical shape, a rectangular shape, a rod shape, a distorted shape, or the like in a planar view. The thickness of the pellets is also not particularly limited. For example, the thickness may be the same as that of a known adsorbent. In particular, the thickness that is applicable to an alcohol concentration device is particularly preferred.

The carbon molecular sieve of the present invention in the form of pellets has a maximum diameter of preferably 0.5 to 5.0 mm. When the maximum diameter is adjusted to be within this range, an alcohol is particularly easily concentrated, processing is easily performed, high strength is achieved, the bulk density can be increased, and application to various uses is easily performed. Furthermore, blockage of piping etc. is unlikely to occur, and application to an alcohol concentration device is easily performed. In the present specification, the term “maximum diameter” refers to an average value of the maximum diameters of 30 randomly collected pieces of the carbon molecular sieve of the present invention.

The carbon molecular sieve of the present invention in the form of pellets has an aspect ratio of preferably 1:1 to 1:5. When the aspect ratio is adjusted to be within this range, an alcohol is particularly easily concentrated, processing is easily performed, high strength is achieved, the bulk density can be increased, and application to various uses is easily performed. Furthermore, blockage of piping etc. is unlikely to occur, and application to an alcohol concentration device is easily performed. The aspect ratio refers to a ratio of the maximum diameter to the minimum diameter of a single piece of the carbon molecular sieve, i.e., the maximum diameter of the carbon molecular sieve/the minimum diameter of the carbon molecular sieve. The aspect ratio refers to an average value of the aspect ratios of 30 randomly collected pieces of the carbon molecular sieve.

In terms of the maximum diameter and the aspect ratio of the carbon molecular sieve of the present invention described above, the maximum diameter and the minimum diameter described later of a single piece of the carbon molecular sieve are measured with a caliper.

The type of alcohol in the alcohol solution to which the carbon molecular sieve of the present invention is applied is not particularly limited. Specific examples of the alcohol include alcohols having 1 to 20 carbon atoms, such as methanol, ethanol, n-propyl alcohol, isopropyl alcohol, n-butyl alcohol, isobutyl alcohol, sec-butyl alcohol, tert-butyl alcohol, n-pentyl alcohol, and n-hexyl alcohol. These alcohols can be used singly or in a combination of two or more.

The carbon molecular sieve of the present invention adsorbs water molecules in an alcohol solution while not adsorbing alcohol, thereby increasing the alcohol concentration. Alcohols having a large number of carbon atoms have a large molecular size; thus, such alcohols do not easily pass through the inlet of the pores of the carbon molecular sieve of the present invention, and are not easily adsorbed on the carbon molecular sieve of the present invention. Accordingly, from the standpoint of easily concentrating an alcohol solution, the alcohol preferably has a large carbon number (e.g., 2 to 20 carbon atoms, and particularly 6 to 20 carbon atoms).

The basis of the present invention lies in the fact that separating water molecules from alcohol is difficult, and the purpose of the present invention has been set to further reduce the energy required for increasing the concentration of alcohol, compared to the energy required in the conventional methods, which use, as an essential requirement, a distillation step to remove water from alcohol vapors. From this standpoint, the present invention is particularly useful because alcohol solutions can be concentrated without performing a distillation step even when the alcohol has a small carbon number (e.g., an alcohol having 1 to 12 carbon atoms, in particular 1 to 6 carbon atoms), although it is particularly difficult to separate such alcohols having a small carbon number from water molecules.

In the carbon molecular sieve of the present invention, the adsorption rate of water molecules is greater than the adsorption rate of the alcohol in an alcohol solution, as described above. In other words, in the carbon molecular sieve of the present invention, the time for adsorbing water molecules is shorter than the time for adsorbing alcohol. Specifically, the ½ equilibrium adsorption time of water molecules may be not more than ½, preferably not more than ⅕, and more preferably not more than 1/10, of the ½ equilibrium adsorption time of alcohol. In this case, the carbon molecular sieve of the present invention has adsorption characteristics of, in particular, having excellent adsorption selectivity for water molecules, and more efficiently increasing the concentration of alcohol. The lower limit is not particularly limited, and a smaller value is more preferred. For example, the ½ equilibrium adsorption time of water molecules may be not less than 1/10000 of the ½ equilibrium adsorption time of alcohol.

In the carbon molecular sieve of the present invention, the ½ equilibrium adsorption time of water molecules is preferably 5 to 600 seconds, and more preferably 6 to 60 seconds. The ½ equilibrium adsorption time of alcohol is preferably 1 to 1500 seconds, and more preferably 10 to 500 seconds. Within these ranges, water molecules are rapidly adsorbed on the carbon molecular sieve of the present invention, making it possible to concentrate an alcohol solution within a short period of time.

In the present specification, the ½ equilibrium adsorption time refers to the time from when the adsorption measurement is started (starting point) to when the adsorption amount becomes 50% of the equilibrium adsorption amount. That is, the ½ equilibrium adsorption time refers to the time from when the adsorption measurement is started (starting point) to when the value of the adsorption amount/equilibrium adsorption amount becomes 0.5.

The carbon molecular sieve of the present invention can be suitably used as a water molecule adsorbent. The water molecule adsorbent comprises the carbon molecular sieve of the present invention, and is thus capable of efficiently concentrating an alcohol solution by efficiently adsorbing water molecules. In particular, by allowing an alcohol solution to pass through a container filled with the carbon molecular sieve of the present invention, the water molecules are more efficiently adsorbed on the carbon molecular sieve of the present invention to thus concentrate the alcohol solution.

The carbon molecular sieve of the present invention described above is used to concentrate an alcohol solution by desorbing water molecules from the alcohol solution. In particular, since the pore-entrance size of the carbon molecular sieve of the present invention is adjusted to be within an appropriate range, the adsorption selectivity is excellent, thus making it possible to efficiently concentrate an alcohol solution. For this reason, the carbon molecular sieve of the present invention can be used as an alcohol concentration material. Therefore, the carbon molecular sieve of the present invention can be suitably used in a method for concentrating an alcohol solution to produce an alcohol with a concentration of, for example, 97 mass % or higher (particularly 99 mass % or higher). In particular, by allowing the alcohol solution to pass through a container filled with the carbon molecular sieve of the present invention, an alcohol solution can be more efficiently concentrated.

The water molecule adsorbent or the alcohol concentration material described above may consist of the carbon molecular sieve of the present invention, or may comprise other known components in combination with the carbon molecular sieve of the present invention.

The carbon molecular sieve of the present invention, which has the above advantages, can be suitably used for an alcohol concentration device.

As long as the alcohol concentration device is provided with the carbon molecular sieve of the present invention, the rest of the configuration may be the same as, for example, those of known alcohol concentration devices.

When a container filled with the carbon molecular sieve of the present invention is provided, for example, as an adsorption column, a feed pump or the like is preferably provided as a means for allowing an alcohol solution to pass through. The relationship between the amount of the carbon molecular sieve of the present invention inserted into the adsorption column and the amount of the alcohol solution introduced in a single adsorption step is preferably set as appropriate. From the standpoint of the concentration of the alcohol solution after being concentrated, the mass of water in the alcohol solution is preferably 5 parts by mass or less (e.g., 0.01 to 5 parts by mass) per 100 parts by mass of the carbon molecular sieve of the present invention. Further, the flow rate of the alcohol solution when passing through (i.e., a value obtained by dividing the flow rate per hour of the alcohol solution by an empty adsorption column) is preferably the following: SV=0.8 to 10 h⁻¹, from the standpoint of the concentration of the alcohol solution after being concentrated. The time for allowing the alcohol solution to pass through is preferably 1 to 60 minutes, from the standpoint of the concentration of the alcohol solution after being concentrated.

Additionally, a diaphragm pump or the like is preferably provided as a means for collecting the alcohol solution (the concentrated alcohol solution) that has passed through the carbon molecular sieve of the present invention in the adsorption column. This allows for more efficient concentration of the alcohol solution.

After concentrating an alcohol solution as described above, the water adsorbed on the carbon molecular sieve of the present invention in the adsorption column is preferably desorbed so as to allow the carbon molecular sieve of the present invention to be reused. The carbon molecular sieve of the present invention can be reused by desorbing the water adsorbed on the carbon molecular sieve of the present invention by heating to 50 to 200° C. Therefore, after concentrating the alcohol solution as described above, a means for heating the inside of the adsorption column is preferably also provided. Typically, there is excess waste heat in factories, and this waste heat is often discharged into the air or water for disposal. By using this waste heat, the water adsorbed on the carbon molecular sieve of the present invention can be desorbed for reuse of the carbon molecular sieve of the present invention with very little running cost. Therefore, after concentrating the alcohol solution as described above, a waste heat inflow pump for introducing waste heat into the adsorption column is preferably provided. The configuration of the device described above is merely an example, and does not limit the present invention.

The alcohol concentration device described above can be used for, for example, chemical synthesis, fuel, and the like.

### 2. Production Method for Carbon Molecular Sieve

The production method for the carbon molecular sieve of the present invention is not limited as long as an carbon molecular sieve, wherein the total volume of pores with an inlet diameter of 0.33 nm or more as determined by a molecular probe method is not less than 3 times the total volume of pores with an inlet diameter of 0.46 nm or more, is obtained. For example, the carbon molecular sieve can be produced by known production methods.

Examples of the production methods for the carbon molecular sieve of the present invention include a heat shrinkage method, an impregnation method, and a chemical vapor deposition (CVD) method. An example of the production method for the carbon molecular sieve of the present invention is described below.

For example, the carbon molecular sieve of the present invention can be produced by a method comprising a carbonization step of carbonizing a carbon precursor to obtain a carbide, and an activation step of activating the carbide.

The carbon precursor is not particularly limited as long as it is a material with which a desired carbon molecular sieve can be obtained.

Examples of carbon precursors include coal, coconut or palm shells (i.e., palm shells, coconut shells, etc.), natural fibers (i.e., hemp, cotton, etc.), synthetic fibers (i.e., rayon, polyester, etc.), synthetic resins (i.e., polyacrylonitrile, phenol resin, polyvinylidene chloride, polycarbonate, polyvinyl alcohol), and charcoal. These carbon precursors can be used singly or in a combination of two or more.

From the standpoint of easily adjusting the pore-entrance size and, if necessary, the pore volume to be within desired ranges, the carbon precursor is preferably coal, coconut or palm shells, synthetic resin, charcoal, or the like. Therefore, the carbon molecular sieve of the present invention is preferably formed of a carbide of at least one material selected from the group consisting of coal, coconut or palm shells, natural fibers, synthetic fibers, synthetic resins, and charcoal, and more preferably formed of a carbide of at least one material selected from the group consisting of coal, coconut or palm shells, synthetic resins, and charcoal.

In the production method for the carbon molecular sieve, the raw material may optionally comprise additives. Examples of additives include water, coal tar, dehydrated tar, hard pitch, coal tar-based pitch, and petroleum-based pitch. The additives can be used singly or in a combination of two or more. The amount of the additive for use may be, for example, 1 to 100 parts by mass per 100 parts by mass of the carbon precursor. When an additive is used, the amount of oxygen in the raw material can be optionally adjusted in advance, for example, to be within the range of 1 to 20 mass %, based on the total amount of the raw material taken as 100 mass %. The amount of oxygen in the raw material can be adjusted, for example, by mixing the carbon precursor and oxygen at 150 to 300° C.

In the production method for the carbon molecular sieve of the present invention, it is also possible to mold the raw material before subjecting it to the carbonization step. For example, carbonization can be performed after the raw material is molded into pellets.

The conditions under which the raw material is carbonized are not particularly limited. For example, the carbon precursor may be carbonized by heating to 300 to 900° C., and more preferably 300 to 800° C., under oxygen-free conditions.

The carbonization time may be appropriately set according to the raw material used and the apparatus for carbonization. For example, carbonization can be performed for 15 minutes to 20 hours, and preferably 30 minutes to 10 hours. The carbonization treatment can be performed using known production apparatus, such as a rotary kiln.

A carbide of the carbon precursor is obtained by performing the carbonization step described above. After carbonization, washing treatment, drying treatment, and the like may be performed. The conditions of these treatments may be, for example, the same as those used conventionally.

The carbide obtained by the above carbonization treatment is activated in the activation step. The activation treatment may be performed by using known methods. For example, an activation method that uses water vapor, oxygen, carbon dioxide gas, or other active gas is appropriately used. For the activation treatment, known production apparatus, such as a rotary kiln and a flow furnace, may be used. For example, activation can be performed by bringing water vapor into contact with the carbide for 1 minute or more at a flow rate of 10 to 300 liters per minute.

The temperature of the activation treatment is not particularly limited. To easily adjust the pore-entrance size and, if necessary, the pore volume to be within desirable ranges, the temperature of the activation treatment is preferably 750 to 1200° C., and more preferably 800 to 1100° C. The partial pressure of the active gas may be 10 to 100%, and preferably 30 to 100%.

The activation time may be adjusted to be within an appropriate range according to the conditions such as the raw material, activation temperature, and production apparatus used, and may be, for example, 0 to 48 hours, and preferably 0.5 to 24 hours. An activation time of 0 hours indicates that activation treatment is not performed. When a synthetic resin is used as the carbon precursor, the carbon molecular sieve of the present invention can be obtained without performing the activation treatment.

After the activation treatment, a heat treatment may be optionally performed. Known methods, including a heat shrinkage method, an impregnation method, a CVD method, etc., are widely used for the heat treatment. In the heat treatment, a carbon source may be used.

In the heat treatment, known carbon sources usable in an impregnation method or a CVD method may be widely used. In the impregnation method, for example, known materials such as coal tar, dehydrated tar, coal tar-based pitch, petroleum-based pitch, and creosote oil (see Japanese Patent No. 4893944) may be widely used as the carbon source. Examples of carbon sources used in the CVD method include alcohols, such as methanol and ethanol; esters, such as ethyl acetate; ketones, such as acetone and methyl ethyl ketone; aromatic hydrocarbons, such as benzene (see JP2004-530622A etc.), toluene, and xylene; hydrocarbons, such as hexane; amide solvents, such as dimethylformamide; and solvents, such as polyhydric alcohols, such as ethylene glycol.

The heat treatment temperature may be 600 to 900° C., and more preferably 700 to 800° C., to easily adjust the pore-entrance size and, if necessary, the pore volume to be within desirable ranges. The heat treatment time can be appropriately determined according to the heat treatment temperature, and may be, for example, 15 to 240 minutes.

The heat treatment can be performed, for example, in a nitrogen or argon atmosphere.

The carbon molecular sieve of the present invention may be obtained by performing the carbonization step, the activation step, and optionally the heat treatment. The thus-obtained carbon molecular sieve of the present invention may be optionally washed by using a known method etc.

In particular, the production method of the present invention is capable of producing the carbon molecular sieve of the present invention by appropriately setting one or more conditions, such as activation conditions, the type and amount of the carbon source for use in the heat treatment (CVD processing temperature).

### EXAMPLES

The present invention is described in more detail below with reference to Examples; however, the present invention is not limited to the embodiments of these Examples.

### Example 1: KP-565

In a rotating bed, 100 parts by mass of coal having a particle size of 0.05 mm or less was placed in an atmosphere of 250° C., and air was circulated so that the oxygen content was 10 mass % based on the total weight of the coal. Next, while adding water to the coal, 25 parts by mass of hard pitch and 15 parts by mass of coal tar were added, and the mixture was kneaded. The outer surface of the mixer was heated to 80° C. so that the mixture was uniformly kneaded. The resulting kneaded product was placed in an extrusion molding machine to be molded into pellets having a diameter of 2.0 mm. The thus-obtained pellets were heated in a rotary kiln for about 5 hours while removing air until the final temperature reached 800° C. Then, water vapor was added at a rate of 100 liters per minute, and the treatment was performed for 30 minutes to obtain an activated product. Subsequently, 2.0 parts by mass of benzene per 100 parts by mass of the obtained activated product was circulated for a period of 120 minutes in a nitrogen atmosphere at 800° C. As a result of this heat treatment, a carbon molecular sieve was obtained.

The shape of the obtained carbon molecular sieve was measured with a caliper, which revealed that the maximum diameter was 1.8 mm, and the aspect ratio was 1:2.

### Example 2: KP-566

A carbon molecular sieve was obtained in the same manner as in Example 1 except that 1.0 part by mass of benzene was used.

The shape of the obtained carbon molecular sieve was measured with a caliper, which revealed that the maximum diameter was 1.8 mm, and the aspect ratio was 1:2.

### Comparative Example 1: KP-567

A carbon molecular sieve was obtained in the same manner as in Example 1 except that benzene was not circulated.

The shape of the obtained carbon molecular sieve was measured with a caliper, which revealed that the maximum diameter was 1.8 mm, and the aspect ratio was 1:2.

### Comparative Example 2: WH2c8/32

Granular Shirasagi WH2c8/32 (activated carbon that is not a carbon molecular sieve) produced by Osaka Gas Chemicals Co., Ltd., was used.

### Test Example 1: Pore-Entrance Size

The pore volumes were determined by the molecular probe method. The pore volume was calculated by measuring the equilibrium adsorption amount using several types of probe molecules with different molecular diameters. Two types of probe molecules were used here: carbon disulfide (0.37 nm) and chloroform (0.46 nm). The numerical values in parentheses are their minimum molecular diameters. The probe molecules were adsorbed on the carbon molecular sieves in an atmosphere of 25° C. and 1 atm, and the pore volumes were calculated from the change in the mass of the carbon molecular sieves after equilibrium adsorption (according to the method disclosed in T. A. Braymer et al., Carbon, Vol. 32, 445-452, 1994). Table 1 shows the measurement results. The results show that the pore volume ratios in Examples 1 and 2 were 3 or more, while the pore volume ratios in Comparative Examples 1 and 2 were less than 3. These results clearly indicate that, in Examples 1 and 2, the total volume of pores with an inlet diameter of 0.33 nm or more as determined by the molecular probe method was not less than 3 times the total volume of pores with an inlet diameter of 0.46 nm or more. The results also suggest that, in Comparative Examples 1 and 2, the total volume of pores with an inlet diameter of 0.33 nm or more as determined by the molecular probe method was less than 3 times the total volume of pores with an inlet diameter of 0.46 nm or more.

TABLE 1

| | | | | Comp. | Comp. |
|---|---|---|---|---|---|
| | | Ex. 1 | Ex. 2 | Ex. 1 | Ex. 2 |
| Pore volume | 0.37 nm or more | 0.188 | 0.196 | 0.204 | 0.600 |
| (cc/g) | 0.46 nm or more | 0.005 | 0.032 | 0.101 | 0.528 |

| | | | | |
|---|---|---|---|---|
| Pore volume ratio | 39.1 | 6.13 | 2.02 | 1.13 |

### Test Example 2: Adsorption Rate

The adsorption rate was measured using a Belsorp-max automatic adsorption device produced by MicrotracBEL Corp. First, 0.01 g of activated carbon for use was placed in a measuring cell, and a pretreatment was performed by heating at 150° C. for 3 hours while reducing the pressure with a rotary pump. Thereafter, the measuring cell was attached to the automatic adsorption device, into which water vapor or ethanol vapor was introduced, and the time required for adsorption was measured. Table 2 shows the results. In Table 2, the term “unmeasurable” means that the value was greater than the measurement limit.

TABLE 2

| | | Ex. 1 | Ex. 2 |
|---|---|---|---|
| ½ adsorption | Water vapor | 0.17 | 0.18 |
| time (min) | Ethanol | Unmeasurable | 1.67 |

| | | |
|---|---|---|
| ½ adsorption time ratio | ∞ | 0.11 |

### Test Example 3

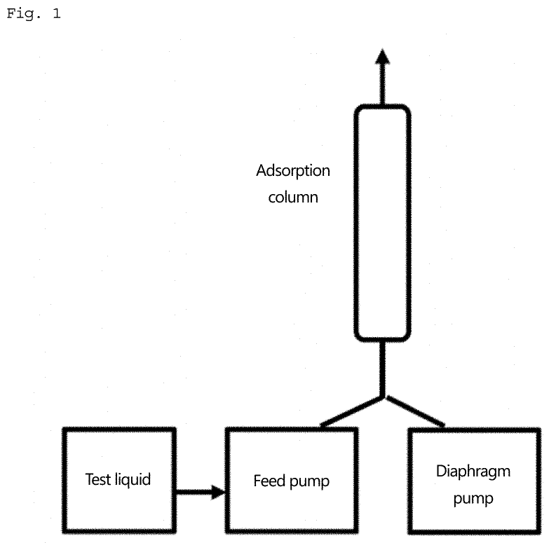

An ethanol concentration test was performed using the ethanol concentration device shown in FIG. 1.

First, carbon molecular sieves for use were washed with water before use to remove fine powder on the surface. Next, 53 g each of the carbon molecular sieves obtained in Examples 1 and 2 were placed in 90-mL adsorption columns. Thereafter, the pressure inside the adsorption columns was reduced, the diaphragm pump was stopped, and 10 mass % of an ethanol solution was fed at 15 mL/min with a feed pump. Subsequently, the liquid that passed through the adsorption column was collected with the diaphragm pump every minute, and the ethanol concentration was calculated from its density. The ethanol concentration was determined by measuring the mass of 5 mL of the collected test liquid.

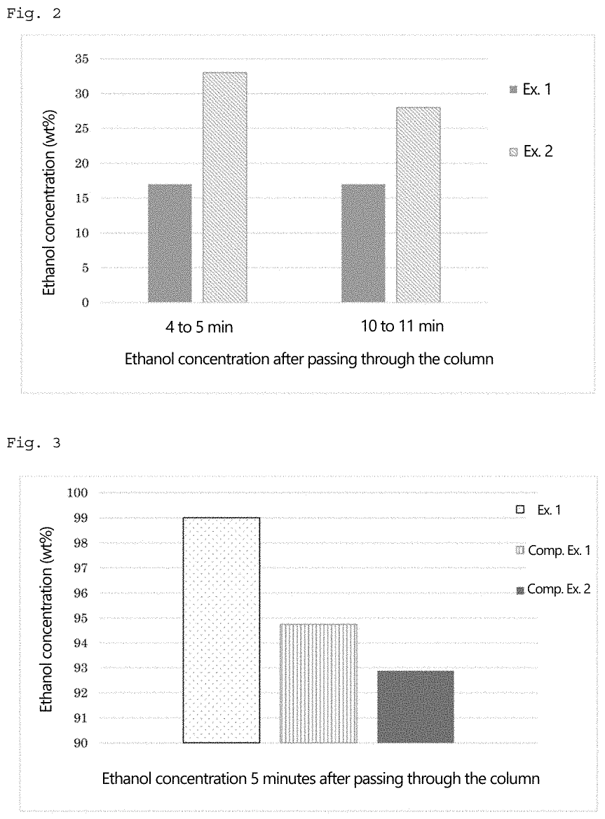

FIG. 2 shows the results. The results confirmed that the ethanol solutions were concentrated. The increase in the ethanol concentration was saturated at some point, followed by a decrease. This is probably because the amount of water molecules reached the capacity of the carbon molecular sieve on which water molecules can be adsorbed, and after that, the concentration relatively decreased by continuously feeding 10 mass % of ethanol. These results suggest that the ethanol solution can be concentrated to a desired concentration, for example, by increasing the amount of the carbon molecular sieve used or by repeating the above concentration step. Further, the concentration of the alcohol solution after being concentrated differed depending of the use of the carbon molecular sieve of Example 1 or 2, which suggests that the results were affected by the pore-entrance size.

### Test Example 4

An ethanol concentration test was performed in the same manner as in Test Example 3 except that the carbon molecular sieves of Examples 1 and Comparative Examples 1 and 2 were used, and an ethanol solution with a concentration of 95 mass % was used.

FIG. 3 shows the results. The results confirmed that the use of the carbon molecular sieves of Comparative Examples 1 and 2 could not increase the concentration to 97 mass % or more even when the time for the alcohol solution was increased. In contrast, the use of the carbon molecular sieve of Example 1 eventually resulted in an increase in the concentration to 99 mass % or more by allowing the solution to pass through for 4 minutes or more.

### Test Example 5

A methanol concentration test was performed in the same manner as in Test Example 3 except that the carbon molecular sieves of Example 1 and Comparative Examples 1 and 2 were used, and a methanol solution with a concentration of 95 mass % was used.

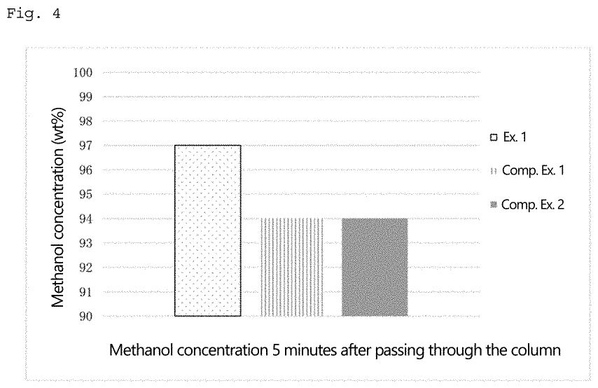

FIG. 4 shows the results. The use of the carbon molecular sieve of Example 1 resulted in an increase in the concentration of methanol, which has a molecular size smaller than that of ethanol. This suggests that separation is possible with lower energy than the energy used in distillation, which is a known method for separating water and methanol.

Accordingly, the use of the carbon molecular sieves having a pore-entrance size within a specific range enabled increasing the concentration of the ethanol solution to 99 mass % or more and the methanol solution to 97 mass % or more. In the present invention, the pore-entrance size of the carbon molecular sieve is sufficiently larger than the size of water molecules but is almost the same as or slightly smaller than the size of alcohol molecules, allowing for selective adsorption of water molecules. Alcohols having a larger number of carbon atoms than ethanol have a larger molecular size than ethanol; thus, it is even more difficult for such alcohols to enter the pores of the carbon molecular sieves of the present invention, compared to ethanol. Therefore, it is clear that the concentration of such alcohols as well can also be increased to 99 mass % or higher.

1. A carbon molecular sieve for adsorbing water molecules in an alcohol solution to separate an alcohol from the water molecules,
wherein
the total volume of pores with an inlet diameter of 0.33 nm or more as determined by a molecular probe method is not less than 3 times the total volume of pores with an inlet diameter of 0.46 nm or more.

2. The carbon molecular sieve according to claim 1, which has a crushed shape, pellet shape, plate shape, rod shape, hollow shape, block shape, honeycomb shape, spherical shape, elliptical spherical shape, distorted shape, or fibrous shape.

3. The carbon molecular sieve according to claim 2, wherein the pellet has a maximum diameter of 0.5 to 5.0 mm and an aspect ratio of 1:1 to 1:5.

4. The carbon molecular sieve according to claim 1, which is a carbide of at least one material selected from the group consisting of coal, coconut or palm shells, natural fibers, synthetic fibers, synthetic resins, and charcoal.

5. The carbon molecular sieve according to claim 1, wherein the alcohol is an alcohol having 1 to 6 carbon atoms.

6. The carbon molecular sieve according to claim 1, wherein the ½ equilibrium adsorption time of the water molecules is not more than ½ of the ½ equilibrium adsorption time of the alcohol.

7. A water molecule adsorbent comprising the carbon molecular sieve of claim 1.

8. An alcohol concentration material comprising the carbon molecular sieve of claim 1.

9. An alcohol concentration device comprising the water molecule adsorbent of claim 7.

10. A method of producing an alcohol with a concentration of 97 mass % or more,
the method comprising bringing the carbon molecular sieve of claim 1 into contact with an alcohol solution.

11. A method for concentrating an alcohol,
the method comprising bringing the carbon molecular sieve of claim 1 into contact with an alcohol solution.

12. A water molecule absorbent comprising the carbon molecular sieve of claim 5.

13. A water molecule adsorbent comprising the carbon molecular sieve of claim 6.

14. An alcohol concentration material comprising the carbon molecular sieve of claim 5.

15. An alcohol concentration material comprising the carbon molecular sieve of claim 6.

16. An alcohol concentration apparatus comprising the water molecule adsorbent of claim 12.

17. An alcohol concentration apparatus comprising the water molecule adsorbent of claim 13.

18. An alcohol concentration apparatus comprising the alcohol concentration material of claim 8.

19. An alcohol concentration apparatus comprising the alcohol concentration material of claim 14.

20. An alcohol concentration apparatus comprising the alcohol concentration material of claim 15.
